(19) United States
(12) Patent Application Publication
(10) Pub. No.: US 2016/0302961 A1
(43) Pub. Date: Oct. 20, 2016

(54) ORAL OCCLUSION DEVICE FOR THE TREATMENT OF DISORDERED BREATHING AND METHOD OF USING SAME

(71) Applicant: Frank E. Seaman, Colorado Springs, CO (US)

(72) Inventor: Frank E. Seaman, Colorado Springs, CO (US)

(21) Appl. No.: 15/096,306

(22) Filed: Apr. 12, 2016

Related U.S. Application Data
(60) Provisional application No. 62/147,612, filed on Apr. 15, 2015.

(51) Int. Cl. A61F 5/56

(52) U.S. Cl. CPC A61F 5/56

(57) ABSTRACT

An oral occlusion device and method for the treatment of respiratory disturbances in human subjects, particularly for the treatment of periodic mouth breathing. An adhesive strip, which in one embodiment is sized to fit the lips within the vermillion border of the lips, gently secures the upper and lower lips to provide gentle oral occlusion, particularly during sleep.

### CROSS-REFERENCE TO RELATED APPLICATIONS

This application claims priority to U.S. Provisional Patent Application 62/147,612; filed Apr. 15, 2015 and which is incorporated as if fully rewritten herein.

### STATEMENT REGARDING FEDERALLY SPONSORED RESEARCH OR DEVELOPMENT

Not applicable.

### TECHNICAL FIELD

The present disclosure relates generally to the field of the treatment of breathing disorders, in particular, to an oral occlusion device and method for the treatment of respiratory disturbance.

### BACKGROUND OF THE INVENTION

Sleep-disordered breathing is an umbrella term for several chronic conditions in which partial or complete cessation of breathing occurs many times throughout the night. Sleep-disordered breathing results in release of stress hormones with daytime sleepiness or fatigue that interferes with a person's ability to function and reduces quality of life. Symptoms may include snoring, pauses in breathing described by bed partners, and increased respiratory effort. Upper airway resistance syndrome, which is by far the most common form of sleep-disordered breathing, is associated with many other adverse health consequences, including an increased risk of death.

To be properly diagnosed, patients with suspected sleep-disordered breathing are often evaluated by a polysomnogram (sleep test), which measures approximately a dozen physiologic parameters during sleep. One of the most important measurements is breathing effort and its cessation during sleep. A breathing pause of 10 seconds or more is generally termed an apnea. Not surprisingly, apneas may be associated with oxygen desaturation (a decrease in blood oxygen) and other bodily responses, as the person struggles to breathe. These arousals may consist of flexing of muscles, including those of the airways, and change in the electrical activity of the brain as measured by an electroencephalogram (EEG). Arousals are complex phenomena that may involve discharges of brain chemicals of the adrenalin family, which may contribute to the health conditions associated with sleep apnea. Desaturation and arousals also occur with hypopnea (partial decrease in air flow). The Apnea-Hypopnea Index is the number of apneas and hypopneas that occur per hour of sleep and is an important measure of the severity of sleep apnea, along with the depth of denaturation.

A single-night polysomnogram in a sleep laboratory can accurately diagnose sleep apnea in most patients. With portable equipment, the diagnosis of sleep apnea is possible in the home setting, and this approach may provide improved access to sleep apnea diagnostic testing. Important indexes from sleep studies are the Apnea/Hypopnea Index (AHI) the Respiratory Disturbance Index (RDI) and oxygen saturations. AHI is a measure that indicates the severity of sleep apnea. It is the average number of apneas and hypopneas per hour of sleep. This is calculated by adding the total number of all apneas and hypopneas and then dividing them by the number of hours the patient spends sleeping. This measure represents the severity of sleep apnea including sleep disturbances and desaturations.

RDI is a measure of the severity of sleep apnea, including sleep disruptions and desaturations. Unlike the AHI, the RDI counts the number of arousals by respiratory effort. It is the average number of sleep disordered events that cause an arousal from sleep per hour of sleep. It is calculated by adding the number of apneas, hypopneas, and respiratory effort related arousals (RERAs) and dividing by the number of hours the patient spends asleep.

Estimates of the prevalence of sleep-disordered breathing vary widely, depending on the methodology. Conservatively, based on laboratory or portable home tests, 4 percent of men, 2 percent of women, and 2 percent of children ages 8 to 11 in the United States have been reported to have sleep-disordered breathing. Other surveys estimate that between 5 and 10 percent of the U.S. adult population have Obstructive Sleep Apnea (OSA); 7 percent have breathing pauses during sleep that put them at risk for more severe sleep events, and 23 to 59 percent snore. Unpublished data from a nationally representative sample of U.S. adults over age 20 show that the symptoms of sleep-disordered breathing (for example, snoring) are more likely to be reported by men than women. From 1980 to 1990, the number of office visits in the United States resulting in a diagnosis of sleep apnea increased from 108,000 to 1.3 million. Despite the increased awareness of sleep-disordered breathing, it has been suggested that 93 percent of women and 82 percent of men with signs and symptoms of moderate to severe sleep-disordered breathing remain undiagnosed.

Factors that have been identified in studies to increase the risk of developing sleep apnea include obesity, male gender, and some ethnic groups (African American, Asian, and Native American). A study of more than 6,400 patients with mild to moderate sleep-disordered breathing found an association between sleep-apnea severity as measured by the Apnea-Hypopnea Index (AHI) and coronary artery disease, heart failure, and stroke. Those with the highest AHI were one-and-a-half times more likely to have had a stroke and more than twice as likely to have heart failure than those with lowest AHI, even when adjusted for other known risk factors, including age, sex, race, body size, hypertension, smoking, and cholesterol levels.

The economic burden of sleep-disordered breathing is significant. Lack of adequate sleep at night for any reason leads to daytime somnolence, and habitual lack of restful sleep can lead to uncontrollable sleep attacks. Sleep-disordered breathing adversely affects daytime alertness and cognition and has been linked to occupational and driving impairment. Sleep apnea has also been shown to increase healthcare utilization. In any assessment of the economic burden of sleep apnea, there are two important considerations: 1) it is highly prevalent in the middle-aged work force, and 2) it contributes to other chronic health conditions, such as heart disease and diabetes, and increases the risk of having a stroke and being in an accident at work or in an automobile.

No single cause of sleep apnea has been identified, although an association with weight and neck size is well known. Causes may include nasal obstruction; mouth breathing; large tonsils (particularly in children); an underactive thyroid gland; the use of alcohol, tobacco, and sedatives; menopause in women; and higher levels of testosterone. Family history and genetic susceptibility studies show that a third of the total variability in sleep apnea severity in populations can be accounted for by heritability or genetic susceptibility. The bony and soft tissue structures of the face, as well as the heritability of obesity, are potential mechanisms by which genetics plays a role in sleep apnea.

A severe form of breathing disorder of sleep is OSA, noted above, which is characterized by recurrent narrowing or collapse of the back of the throat because of the loss of muscle tone that occurs during sleep. A less common form, central sleep apnea, is distinguished by cessation of breathing efforts during sleep. There is no struggle to breathe; the brain just does not send the proper breathing signals. Both result in repetitive events of insufficient air flow, oxygen absorption, and carbon dioxide exhalation. Reduction in blood oxygen levels may lead to a hormonal stress response by the body. This reaction may arouse, but not fully awaken, the sleeper, who repeats the events with the next period of sleep. If the cycle of arousals is repeated many times during the night, a cascade of stress-hormone release ensues, which is thought to be responsible for many of the adverse health consequences associated with sleep-disordered breathing. A very common form of increased effort to breath is upper airway resistance syndrome (UARS). This is a sleep-related breathing disorder in which repetitive increases in resistance to airflow in the upper airway lead to brief arousals (RERAs) and daytime fatigue. It is usually associated with loud snoring. Obstructive Sleep Apnea (OSA), characterized by apneas and hypopneas, may be totally absent. Although blood oxygen levels may be in the normal range, the patient can still have symptoms of Obstructive Sleep Apnea, e.g., excessive daytime sleepiness. This is a result of the stimulation of sleep arousals and their repetitive release of stress hormones.

The upper airway in patients with OSA is often smaller than normal. It may be narrowed by fat deposition in obese individuals or other structural factors, such as airway length, position of the jaw, or size of the tongue. A narrowed air passage can collapse more frequently and completely when the muscles of the throat, which keep the upper airway open during wakefulness, relax during sleep. Changes in body position and the reduced lung expansion that occur with sleep interact with these other factors and may lead to further upper airway vulnerability.

Experimental animal studies as well as observations in patients with central sleep apnea show that the brain centers responsible for the control of rhythmic respiratory muscle activity are more unstable compared to people without this disorder. Some individuals may have both obstructive and central sleep apnea. For children suffering from sleep apnea, surgical treatment with removal of tonsils (and adenoids) is the first choice. However, the long-term effects of this procedure on sleep-disordered breathing in these children are poorly understood.

There is considerable variation in the severity of sleep apnea from night to night, depending upon duration of sleep, body position, time spent in different stages of sleep, and other factors, such as alcohol consumption before going to bed. Alcohol and certain sleeping medications may cause deeper relaxation of the airways during sleep and a blunting of the sleeper's arousal response, thus allowing longer and more frequent apneas and greater desaturations.

Prevention of weight gain and obesity is critical for reducing the risk of developing clinically significant OSA. Appropriate evaluation and treatment of any nasal passage obstruction is important in reducing the collapsibility of the upper airway. Smoking cessation should be pursued by all patients. Avoiding alcohol and sedatives and developing better sleep hygiene may be helpful.

Physicians use the Apnea-Hypopnea Index (AHI) to assess the severity of sleep apnea based on the number of complete cessations of breathing (apnea) and partial obstructions (hypopnea). Although the Apnea-Hypopnea Index (AHI) is interpreted in the context of the patient's symptoms, age, and other medical conditions, an Apnea-Hypopnea Index (AHI) of more than 5 with symptoms is generally abnormal enough to warrant treatment. As the condition is usually chronic, in the absence of significant modification of a risk factor, the treatment prescribed should be used long term.

Treatments for OSA work by physically increasing the size of the upper airway. A very effective treatment is a continuous positive airway pressure (CPAP) device that delivers pressurized air to the upper airway, via a mask, splinting the airway open. However, the effectiveness of this treatment is often substantially reduced or nullified by inconsistent or inadequate use by patients. Professionally assisted adjustments of the mask size and type, the addition of humidity, and the treatment of nasal congestion and blockage may improve the ability to use this treatment.

There is no presently available effective and safe drug treatment for sleep apnea. External and intra nasal dilators improve snoring, but their efficacy in reducing sleep-disordered breathing has not been adequately shown by controlled trials. In certain patients, surgical treatment or dental devices may be effective, but more studies are needed.

Although breathing abnormalities that occur during wakefulness and sleep have been reported since the 1800s, the high prevalence of disordered breathing that occurs only during sleep was not recognized until 1993. The risk factors for sleep-disordered breathing and the high prevalence of sleep apnea, as well as the adverse health conditions associated with untreated sleep apnea, including increased mortality, have been identified by multiple large-scale observational studies. There is, however, an urgent need for large-scale clinical studies to determine the natural course and benefit of treatments on the longer-term health in people with all levels of sleep-disordered breathing, especially with regard to its severity, effect on cardiovascular health, and survival.

Given the remarkable rise of obesity and the high prevalence of diabetes today, it would also be important to learn the effects of these conditions on the course and treatment of sleep apnea. Intervention at early stages has the potential to become an effective prevention strategy. Confirmation of whether portable and home-based diagnostic monitoring and auto-adjusting therapeutic CPAP devices could adequately supplement formal laboratory-based evaluation, and, if so, in which populations, would lead to more cost-effective healthcare delivery. Studies thus far support the use of oral appliances in mild to moderate sleep-disordered breathing and the use of surgery primarily as adjunctive treatment for adults or in “CPAP failures.” Electrical stimulation of the nerves to activate the upper airway muscles and dilate the airway has been associated with beneficial effects on sleep-disordered breathing, but this approach needs further study to determine efficacy as well as the design of equipment for clinical use.

It is as yet not clear whether the candidate genes for sleep apnea (for example, the APOE epsilon gene) lead directly to sleep apnea or if these genes are linked to intermediate factors that increase the risk of sleep apnea via their effects on other factors, such as facial structure and obesity. Future studies involving analysis of multiple genes simultaneously in well-defined subgroups of persons with sleep apnea hold the promise for development of predictive models that will enable early diagnosis and intervention in the appropriate populations. A genetic approach also may lead to better understanding of the basic mechanisms of the condition, which is a prerequisite for the development of future therapies.

### SUMMARY OF THE INVENTION

Multiple embodiments of an oral occlusion device for the treatment of disordered breathing, in particular, for the treatment of periodic mouth breathing, are described. In a basic embodiment, such oral occlusion devices may have a first surface a first surface length, a first surface width, and a first surface thickness. The oral occlusion device may also have a second surface, a second surface length, a second surface width, a second surface thickness, and an adhesive area (210).

The oral occlusion device many be manufactured in many ways. The first surface and second surface may represent two sides of a unitary structure, or may be formed of two or more joined layers. One skilled in the art will be able to choose appropriate materials and dimensions to accomplish goals such as flexibility, durability, adhesiveness, and dimensions so as to removably, securely, and comfortably hold the upper and lower lips in at least a predominantly closed orientation.

The overall shape of the device may vary, and in particular, may be formed so as to have a similar shape to the overall shape of a patient's peri-oral area, and of course may be sized accordingly.

In some embodiments, the second surface may have a central non-adhesive area bounded by the adhesive area. Such an embodiment would allow the device to be adhered to the peri-oral skin without the necessity of adhesion to the lip tissue itself, which is much more prone to trauma break-down due to repeated tape adhesions. The central non-adhesive area may include a central non-adhesive dressing area, which could be formed of a gauze area, a gel area, or of any other suitable non-adhesive material that would be known by one skilled in the art. The central non-adhesive dressing area may also include a medicament, which could range across a wide variety of substances that would be known by one skilled in the art, including by way of example and not limitation, lubricants, antibiotics, drugs, flavorings, and many other substances.

In yet other embodiments, the second surface may have at least one peripheral non-adhesive area. Such a peripheral non-adhesive area could aid in giving the user something to pull on to gently detach the device and may include at least one removal fixture, which could be a humanly-grippable tab, again to aid the user in peeling the device away from the peri-oral area after use.

To preserve the adhesive qualities of the second surface the device may be packaged prior to use with a protective cover removably adhered to the second surface that may be removed by the patient prior to use. The entire device, including any protective cover, may be provided suitably sealed in a sterile, or at least clean, outer packaging.

In another embodiment an oral occlusion device for the treatment of disordered breathing may be sized and shaped to be adhered to an area of lip skin within a vermillion border of a human user's lips. Lip skin is a transitional skin separating the moist mucosal tissue of the mouth and the dry facial skin epidermis. The vermillion border represents the change in the epidermis from highly keratinized external skin to less keratinized internal skin. In such an embodiment, adequate functionality has been demonstrated by adhering to the lip skin while avoiding redundant facial skin contact, which can be uncomfortable for the patient. As one skilled in the art would realize, the oral occlusion device could be made in various sizes and shapes so as to closely fit the particular individual's lip shape of users.

### BRIEF DESCRIPTION OF THE DRAWINGS

Without limiting the scope of the oral occlusion device as disclosed herein and referring now to the drawings and figures:

FIG. 1 is a top plan view of an embodiment of an oral occlusion device;

FIG. 2 is a side view of the oral occlusion device of FIG. 1;

FIG. 3 is a bottom plan view of the embodiment of FIG. 1;

FIG. 4 is a bottom plan view of an embodiment of an oral occlusion device;

FIG. 5 is a side view of the embodiment of FIG. 4; and

FIG. 6 is a front view of an embodiment of the present invention applied to the lips of a human subject, the subject being shown in broken line form.

These drawings are provided to assist in the understanding of the exemplary embodiments of the oral occlusion device as described in more detail below and should not be construed as unduly limiting such a device. In particular, the relative spacing, positioning, sizing and dimensions of the various elements illustrated in the drawings are not drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

Mouth breathing (also termed open-mouth breathing or mouth breathing habit) is breathing through the mouth rather than the nose. Human infants are sometimes considered obligate nasal breathers, but generally speaking healthy humans may breathe through their nose, their mouth, or both. During rest, breathing through the nose is common for most individuals. Breathing through both nose and mouth during exercise is also normal, a behavioral adaptation to increase air intake and hence supply more oxygen to the muscles. Mouth breathing may be called abnormal when an individual breathes through their mouth even during rest. Some sources use the term “mouth breathing habit” but this incorrectly implies that the individual is fully capable of normal nasal breathing, and is breathing through their mouth out of preference. However, in many cases, mouth breathing represents an involuntary, subconscious adaptation to reduced patency of the nasal airway, and mouth breathing is a requirement simply in order to get enough air.

Mouth breathing has been demonstrated to be associated with reduction of the retropalatal, retroglossal areas of the upper airway, and lengthening of the pharynx. The faster airflow generated by the longer and narrower upper airway may increase the negative intraluminal pressure during inspiration and thereby facilitate the collapse of the upper airway. (See: Lee et al: How does open-mouth Breathing Influence Upper Airway anatomy? Laryngoscope 117; June 2007.) Mouth breathing is associated with oral function as well. It promotes lip incompetence.

It promotes a lower tongue position and tongue thrusting with swallowing. It also promotes dry mouth and pharyngeal tissue. These desiccated tissues have increased inflammation, increased swelling and increased stickiness, and can also promote airway collapse and respiratory disturbance with increased effort.

It has been demonstrated during development of some of the embodiments of the present invention that unobstructed nasal breathing during sleep causes significantly fewer RERAs and lower RDIs than habitual mouth breathing during sleep.

A goal of treatment is, therefore, to promote an environment to breathe through the nose, if it is unobstructed. Deviated septums and enlarged turbinates may need to be evaluated and treated.

Many patients also suffer from sleep bruxism (SB). Sleep bruxism (SB) has historically been treated as an isolated oral issue. Recently however, research has shown a correlation between Sleep Bruxism (SB) and sleep arousals in patients with Sleep-Disordered Breathing (SDB). Sleep-Disordered Breathing (SDB) is defined as abnormalities in respiratory patterns or ventilation frequency during sleep. It pertains to 1) Upper Airway Resistance Syndrome, 2) Obstructive Sleep Apnea, and 3) Central Sleep Apnea. Hypoxia results from obstruction (a ventilatory problem) or improper respiratory pattern and blood gases/pH problems (an arousal problem).

Mouth breathing has been classified according to etiology into 3 groups: obstructive, habitual and anatomic. The nasal airway may be compromised partially, where there is increased resistance to the flow of air due to narrowing of the lumen at some point in the upper respiratory tract, or completely obstructed. Such individuals may find it difficult or impossible to breathe through their nose alone. Specific causes of nasal obstruction which have been linked to mouth breathing include antrochoanal polyps. Chronic mouth breathing in children may have implications on dental and facial growth. It also may cause gingivitis (inflamed gums) and halitosis (bad breath); especially upon waking if mouth breathing occurs during sleep.

“Pregnancy rhinitis” may lead to nasal obstruction and mouth breathing. This tends occur in the third trimester of pregnancy. In other cases, the upper lip may be short, and the lips do not meet at rest (“lip incompetence”). Gingivitis, gingival enlargement, and increased levels of dental plaque are common in persons who chronically breathe through their mouth. The usual effect on the gums is sharply confined to the anterior maxillary region, especially the incisors (the upper teeth at the front). The appearance is erythematous (red), edematous (swollen) and shiny. This region receives the greatest exposure to airflow during mouth breathing, and it is thought that the inflammation and irritation is related to surface dehydration, but in animal experimentation, repeated air drying of the gums did not create such an appearance.

It has been suggested that chronic mouth breathing in children can lead to the development of a long, thin face, sometimes termed “long face syndrome,” or specifically “adenoid faces” when the mouth breathing is related to adenoid hypertrophy. Malocclusion of the teeth (e.g. “crowded teeth”) is also suggested to result from chronic mouth breathing in children. Conversely, it has been suggested that a long thin face type, with corresponding thin nasopharyngeal airway, predisposes to nasal obstruction and mouth breathing, i.e., a long thin face may cause mouth breathing rather than the other way around. Facial form is also strongly influenced by genetic factors. The following other conditions are also associated with mouth breathing: cheilitis glandularis, Down syndrome, anterior open bite, tongue thrusting habit, cerebral palsy, sleep apnea and snoring.

Some individuals breathe through their mouth through force of habit, perhaps due to a previous cause of nasal obstruction that is now corrected. This is of significance to the present invention, as it has been found that simple occlusion of mouth breathing may cause the mouth-breather to revert back to normal nasal breathing patterns.

In can be helpful, before beginning treatment, to assess a prospective patient's Respiratory Disturbance Index (RDI), beginning with the following, or one similar to it, multipart subjective screening questionnaire:

Prospective patients may be asked to answer the following screening information.
1. Are you able to passively breath through your nose for extended periods of time?
2. Are you aware of breathing through your mouth at night?
3. Are you thirsty during the night or wake up needing a glass of water?
4. Do you smoke?
5. Do you grind or clench your teeth at night?
6. Do you wake up with jaw fatigue or temple headaches?
7. Are you tired when you wake up?
8. Are you a light sleeper?
9. How would you rate your average night's sleep on a scale from 1-10?
10. Are you awakened by children, dogs or a snoring partner?
11. Do you have nasal congestion or allergies at night?
12. Do you use nasal rinses prior to sleep?
13. In the past or the present, have you had the need of supplemental oxygen or a CPAP (Continuous Positive Airway Pressure) during sleep?

Following a subjective report suggestive of periodic mouth breathing at night, RDI testing helps to evaluate the effectiveness of nose breathing versus periodic mouth breathing at night. RDI software continuously records oxygen and heart rate every 3 seconds, all night long, throughout the extent of the testing-period. Computer designed algorithms convert the patterns of fluctuation of oxygen and heart rate into breathing disturbances (called Respiratory Disturbances). One can then analyze the respiratory disturbances of mouth breathing. Certain variables have a strong impact on these comparisons. Nose restrictions such as an undiagnosed deviated septum will have higher respiratory disturbance numbers with breathing restricted to the nose, by means of an oral occlusion device over the lips. A mouth breathing habit will cause dry mouth and throat, which produce respiratory interruptions (disturbances), Other screening data obtained are a complete profile of oxygen levels, heart rate and fluctuations and obstructive nasal breathing. For best RDI test results, patients are instructed to refrain from stimulants 4 hours prior to sleep (i.e., alcohol, drugs, caffeine, sugar) and to sleep on their side.

On the first night, patients are asked to measure their oxygen saturations during their normal sleeping routine, without an oral occlusion device. On a consecutive night, they are asked to make the same measurements with an oral occlusion device in place.

In particular, it has been found that occlusion of mouth breathing by the application of oral occlusion device to the lips during sleep may be helpful in causing such a reversion to normal nasal breathing patterns. One type of oral occlusion device may be a flexible adhesive strip that is placed horizontally across the upper and lower lips to prevent airflow through the mouth, as will be discussed below.

The product has a number of purposes, by way of example and not limitations only, include:

1. Promote a healthy breathing pattern in which inspiration and expiration occurs solely through the nose.

2. Detect problems with nasal restrictions and congestion.

3. Decrease sleep arousal frequency.

4. Reduce nocturnal sympathetic nervous system hyperactivity.

5. Reduce respiratory disturbances by improving respiratory effort in the effort to breath.

Benefits of use include, by way of example and not limitations only, include:

1. Improved airflow to the lungs as compared with mouth breathing.

2. Reduction of hyperventilation.

3. Hydration of the air entering the pharynx and lungs. This promotes less resistance to the airflow.

4. Prevention of dry mouth and its complications including gingivitis, cavities and halitosis.

5. Reduction in snoring.

6. Improvement in pulse oxygenation.

7. Reduction in sleep arousals, sleep bruxism, TMD pain, teeth pain and root canals, teeth wear.

8. Reduction in sympathetic tone and hypertension.

9. Reduction in asthma.

10. Reduction in cellular inflammation and associated chronic pain.

11. Reduction in oral developmental problems.

12. Reduction of reverse tongue position and swallowing problems.

In a common embodiment, a plastic tape is applied across the lips, causing the lips to remain closed during sleep. One embodiment comprises a section of tape that is approximately one inch longer than the closed lips are wide, allowing for about one half inch of lateral anchorage. The width of the tape should be selected so as to sufficiently extend above the upper lip and below the lower lip so that air leakage around the tape is minimized. Various tapes are suitable for use, although surgical-type tape and those that are particularly formulated for minimal skin reactivity are preferred. The tape may be applied just before sleep, and removed by the patient upon awakening. Any standard sleep study may be performed with the tape in place.

Common results of usage, by way of example and not limitation only, include

1. The oral occlusion device can be used during sleep for a more restful night's sleep.

2. The oral occlusion device is also used to help promote natural, habitual nasal breathing.

3. The oral occlusion device also can be used while exercising.

4. Reduces respiratory disturbances and sleep arousals.

5. Reduces over-ventilation.

6. Improves end-tidal carbon dioxide (ETCO₂) retention promote a lower blood PH and oxygen release from hemoglobin.

In another series of embodiments, the role of an oral occlusion device may be taken by any appliance that causes a functional obstruction to mouth breathing. These may include many types of orthotics or prosthetics, as well as numerous appliances that may be applied to the head or face area, so long as a functional, and easily reversible, obstruction to mouth breathing is effected.

Clinical results using the product (formal research protocols not yet in place) are ongoing to assess the role of an oral occlusion device in treating sleep bruxism (SB) and sleep-disordered breathing (SDB). The Minolta PULSOX 300i pulse-oximeter may be used to gather comparison oxygenation data in mouth breathing vs. nasal breathing. The PULSOX 300i records data every 3 seconds and has sophisticated software to make objective comparisons. Data on RERAs and RDI, pulmonary ventilation and pulse oxygenation, interruptions in breathing, and sleep arousals are detected and compared. Preliminary findings show that use of an oral occlusion device, in the absence of nasal obstruction, results in significantly improved oxygenation and/or fewer sleep arousals.

By way of example and not limitation, a clinical trial of the oral occlusion device was performed in seven patients, with the following results:

### Example 1

Seven current Temporomandibular Disorder (TMD) patients, in a prosthodontic dentistry practice, with signs and symptoms of mouth breathing, dry mouth, low tongue posture, lip incompetence and ability to nose breathe were treated as above. All testing was done between Jun. 1, 2013 and Jan. 26, 2016. All the patients had demonstrated the ability to breathe without restriction through the nose before treatment.

TABLE 1
7 Patient Respiratory Disturbance Index (RDI), comparing Mouth vs. Nose Breathing

| | | | Treatment | | | |
|---|---|---|---|---|---|---|
| | Pre-Treatment (RDI) | | Duration | Post-Treatment (RDI) | | |
| | No Tape | Tape | Months | No Tape | Tape | Improvement (%) |
| #1 50 year-old female | 12 | 6 | 16 | 8 | 3 | 75% |
| #2 50 year-old female | 9 | 5 | 15 | 2 | 0 | 100% |
| #3 71 year-old female | 26 | 10 | 18 | 11 | 4 | 85% |
| #4 67 year-old female | 13 | 2 | 25 | 7 | 3 | 77% |
| #5 21 year-old male | 18 | 8 | 17 | 5 | 0 | 100% |
| #6 28 year-old male | 9 | 5 | 12 | 6 | 3 | 67% |
| #7 60 year-old male | 48 | * | 35 | 11 | 14 | * |
| | | | 19.7 months | | | 82.1% |
| | | | average | | | Improvement |

(* = Not measured)

Patients were an average age of 49.6 years old and all wore the oral occlusion device at night, for an average of 19.7 months. Respiratory Index is the combined total of Respiratory Effort Related Arousals (RERA's) and Oxygen Desaturation Index>4 (0D14) per hour. These demonstrate interruptions in breathing. Interruption in breathing results from increased resistance to airflow and increased mucosal tissue surface tension. Common causes are dry mucosal tissue, tongue obstruction/position and nasal obstructions and congestion.

There was an 82.1% average improvement with breathing disturbances while breathing through the nose during sleep. Improved breathing results in less sleep arousals and improved oxygen uptake and sleep. An oral occlusion device provides a means to keep the lips comfortably closed during sleep and restrict the breathing to nose breathing.

The nightly use of an oral occlusion device as demonstrated in Table 1 above, aids in a significant reduction in respiratory disturbances which is a measure of the severity of the Respiratory Disturbance Index and Obstructive Sleep Apnea. Respiratory disturbances include Respiratory Effort Related Arousals (RERAs) and Apnea/Hypopnea Index (AHI). Both stimulate an arousal from sleep with similar arousal characteristics of cardiac/autonomic nervous system activation, stress hormone (glucocorticoid and epinephrine) release, and rhythmic masticatory muscle (RMMA) activity. The RMMAs facilitate an attempt to open the upper airway which also occur with several deep breathes and a swallowing event. Restriction of breathing through the mouth during sleep has been demonstrated to reduce these occurrences. This has many profound benefits: reduction of airway collapse, improved sleep, reduced sympathetic nervous system activity, and reduction in RMMAs and sleep bruxism. Harmful effects of sleep bruxism include teeth grinding and clenching at night with consequent teeth damage and pain, and morning headaches.

A further advantage is a significant reduction in oral and pharyngeal mucosal dryness. Mouth breathing has a drying effect on the aqueous nature of mucosal tissue and changes its mucopolysaccharide surface characteristics from a slimy surface to a sticky surface. This sticky lining of the oral and pharyngeal structures increases its surface tension, stickiness to one another, increases airflow resistance, and increases bacterial and pollen adherence. It also stimulates the urge of thirst. Other advantages of the oral occlusion device, by way of example and not limitation only, are that its use improves the position and function of the tongue, reduces snoring during sleep, reduces nasal congestion formation, improves nitric oxide transport from the maxillary sinuses to the lungs, reduces the habit of mouth breathing, and results in reduced dental and oral tissue breakdown.

The oral occlusion device is a simple device which is affordable by the masses with profound benefits to those who suffer the harmful consequences of nocturnal mouth breathing. It is designed to be the most comfortable, flexible, have minimized porosity, have a least effective adhesiveness consistent with adequate adhesion, and may, in at least one embodiment described below, be shaped in the configuration of the lips in a closed position. Such a lip-shaped embodiment keeps the adhesive confined to the lips and helps with unnecessary facial skin sensitivity and discomfort. It also allows effective usage by males with facial hair around the lips.

As would immediately recognized by one skilled in the art, the oral occlusion device is not indicated for people who have severe obstructive sleep apnea, fragile health, or an obstructive nasal airway.

As one skilled in the art would know, a very wide range of materials may be suitable for the construction of the oral occlusion device. General requirements are that the device be soft, pliable and comfortable on the lips. In particular, a valuable material has proven to be 3M™ (3M Corporation, St. Paul, Minn., USA) Product Number 2467P, a spunlace, polyester, nonwoven tape with a silicone adhesive on a liner. It is to be emphasized that this is only one material, and one skilled in the art will be able to envision many other materials that would be suitable for the construction of the oral occlusion device.

The product has undergone significant testing by the manufacturer which reports as follows. The adhesive used in Product 2476P was subjected to the several safety evaluations. The product was tested for in vitro cytotoxicity to determine the potential for cytotoxicity based on the requirements of International Organization for Standardization (ISO 10993-5) Biological Evaluation of Medical Devices—Part 5 Tests for In Vitro Cytotoxicity. Triplicate wells were dosed with a 1 cm×1 cm portion of the test article. Triplicate wells were dosed with a 1 cm length of high density polyethylene as a negative control. Triplicate wells were dosed with a similar portion of latex as a positive control. Each was placed on an agarose surface directly overlaying a sub-confluent monolayer of L-929 mouse fibroblast cells. After incubating at 37 degrees C. in the presence of 5% CO2 for 24 hours, the cultures were examined macroscopically and microscopically for any abnormal cell morphology and cell lysis. The test article showed no evidence of causing mild cell lysis or toxicity. The test article met the requirements of the test since the grade was less than a grade 2 (mild reactivity).

A MEM elution in vitro study was conducted to evaluate for potential cytotoxic effects following the guidelines of International Organization for Standardization 10993-5 Biological Evaluation of Medical Devices, Part 5 Tests for In Vitro Cytotoxicity. A single preparation of the test article was extracted in single strength Minimum Essential Medium at 37 degrees C. for 24 hours. The negative control, reagent control and positive control were prepared similarly. Triplicate monolayers of L-929 mouse fibroblast cells were dosed with each extract and incubated at 37 degrees C. in the presence of 5% CO2 for 48 hours. Following incubation, the monolayers were examined microscopically for abnormal cell morphology and cellular degeneration. The test article extract showed no evidence of causing cell lysis or toxicity. The test article met the requirements of the test since the grade was less than a grade 2 (mild reactivity).

The test article was also evaluated, in vivo, for primary skin irritation in accordance with the guidelines of ISO 10993 Biological Evaluation of Medical Devices—Part 10 Tests for Irritation and Delayed-Type Hypersensitivity. Two 25 mm×25 mm sections of the test article and control article were topically applied to the skin of each of three rabbits and left in place for 24 hours. The sites were graded for erythema and edema at 1, 24, 48, and 72 hours after removal of the single sample application.

There was no erythema and no edema observed on the skin of the animals. The Primary Irritation Index for the test article was calculated to be 0.0. The response of the test article was categorized as negligible.

The test article was further evaluated in vivo for the potential to elicit delayed dermal contact sensitization in the guinea pig based on the requirements of ISO 10993-10, Biological Evaluation of Medical Devices, Part 10: Tests for Irritation and Skin Sensitization. The test article was extracted in 0.9% sodium chloride USP and sesame oil, NF. Each extract was intradermally injected and occlusively patched to ten test guinea pigs (per extract). The extraction vehicle was similarly injected and occlusively patched to five control guinea pigs (per vehicle). Following a recovery period, the ten test and five control animals received a challenge patch of the appropriate test and vehicle control. All sites were observed for evidence of dermal reactions at 24 and 48 hours after patch removal. The test article showed no evidence of causing delayed dermal contact sensitization in the guinea pig. The test article was not considered to be a sensitizer in the guinea pig maximization test.

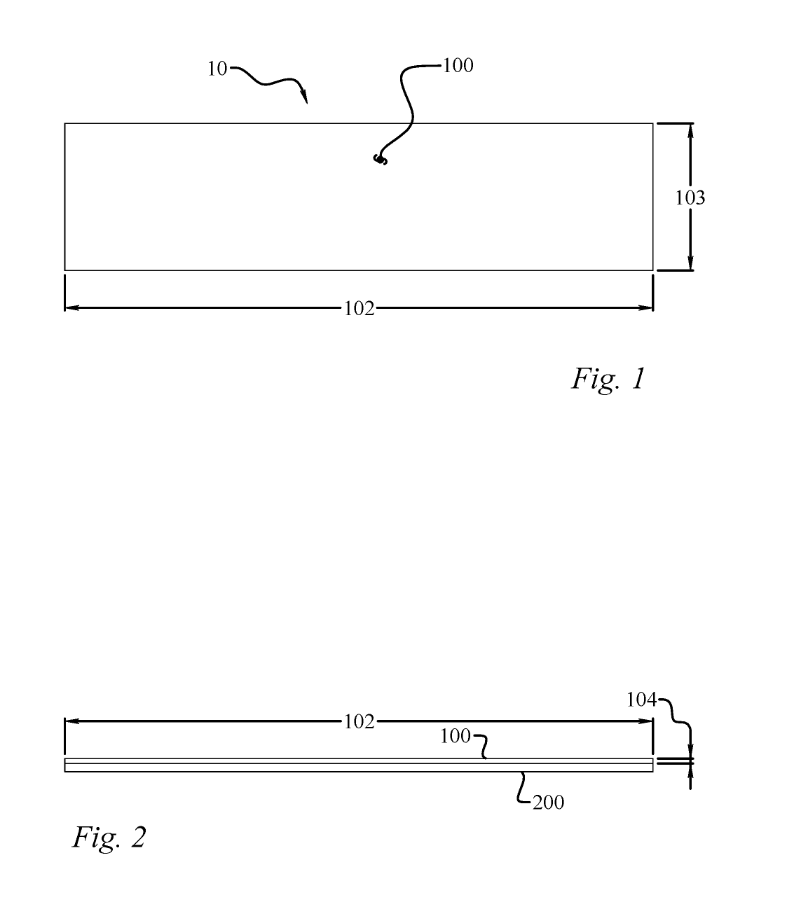

What is claimed, then, as seen in certain embodiments in FIGS. 1-6, is an oral occlusion device (10) for the treatment of disordered breathing, in particular, for the treatment of periodic mouth breathing. As seen well in FIGS. 1 and 2, such oral occlusion devices (10) may have a first surface (100) having a first surface length (102), a first surface width (103), and a first surface thickness (104). As seen well in FIGS. 2 and 3, the oral occlusion device (10) may also have a second surface (200), having a second surface length (202), a second surface width (203), a second surface thickness (204), and an adhesive area (210). The first surface (100) and second surface (200) may represent two sides of a unitary structure, or may be formed of two or more joined layers. One skilled in the art will be able to choose appropriate materials and dimensions to accomplish goals such as flexibility, durability, adhesiveness, and dimensions so as to removably, securely, and comfortably hold the upper and lower lips in at least a predominantly closed orientation. As may be inferred from FIGS. 3 and 4, the overall shape of the device (10) may vary, and in particular, may be formed so as to have a similar shape to the overall shape of a patient's peri-oral area, and of course may be sized accordingly.

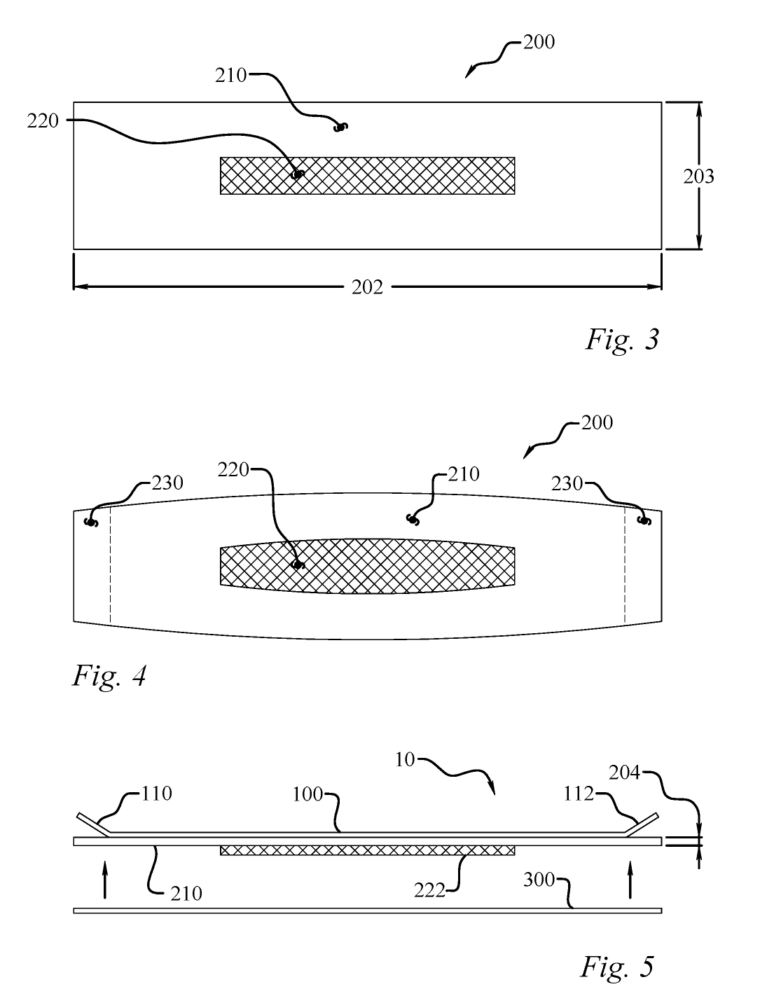

Again with reference to FIGS. 3 and 4, the second surface (200) may a central non-adhesive area (220) bounded by the adhesive area (210). Such an embodiment would allow the device to be adhered to the peri-oral skin without the necessity of adhesion to the lip tissue itself, which is much more prone to trauma break-down due to repeated tape adhesions. As seen well in FIG. 5, the central non-adhesive area (220) may include a central non-adhesive dressing area (222), which could be formed of a gauze area, a gel area, or of any other suitable non-adhesive material that would be known by one skilled in the art. The central non-adhesive dressing area (222) may also include a medicament, which could range across a wide variety of substances that would be known by one skilled in the art, including by way of example and not limitation, lubricants, antibiotics, drugs, flavorings, and many other substances.

As seen well in FIG. 4, the second surface (200) may have at least one peripheral non-adhesive area (230). Such a peripheral non-adhesive area (230) could aid in giving the user something to pull on to gently detach the device (10) from the peri-oral area. In a similar fashion, the first surface may include at least one removal fixture (110), seen well in FIG. 5, which in some embodiments might be configured as at least one humanly-grippable tab (112), again to aid the user in peeling the device (10) away from the peri-oral area after use.

To preserve the adhesive qualities of the second surface (200) the device (10) may be packaged prior to use with a protective cover (300) removably adhered to the second surface (200), seen well in FIG. 5, that may be removed by the patient prior to use. The entire device (10), including any protective cover, may be provided suitably sealed in a sterile, or at least clean, outer packaging.

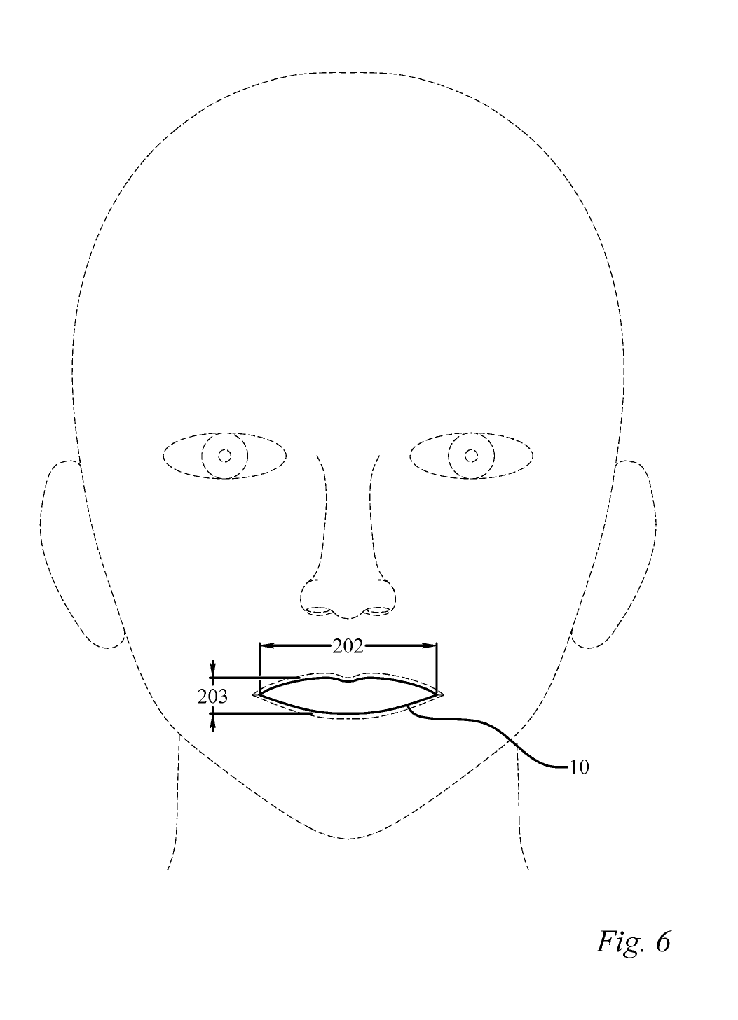

In another embodiment an oral occlusion device (10), seen well in FIG. 6, for the treatment of disordered breathing may be sized and shaped to be adhered to an area of lip skin within a vermillion border of a human user's lips. Lip skin is a transitional skin separating the moist mucosal tissue of the mouth and the dry facial skin epidermis. The vermillion border represents the change in the epidermis from highly keratinized external skin to less keratinized internal skin. In such an embodiment, adequate functionality has been demonstrated by adhering to the lip skin while avoiding redundant facial skin contact, which can be uncomfortable for the patient. As one skilled in the art would realize, the oral occlusion device could be made in various sizes and shapes so as to closely fit the particular individual's lip shape of users.

The presently disclosed oral occlusion device enables a significant advance in the state of the art. The preferred embodiments of the oral occlusion device accomplish this by new and novel arrangements of elements and methods that are configured in unique and novel ways and which demonstrate previously unavailable but preferred and desirable capabilities. The description set forth herein is intended merely as a description of the presently preferred embodiments of the oral occlusion device, and is not intended to represent the only form in which the oral occlusion device may be constructed or utilized. The description sets forth the designs, functions, means, and methods of implementing the oral occlusion device in connection with the illustrated embodiments. It is to be understood, however, that the same or equivalent functions and features may be accomplished by different embodiments that are also intended to be encompassed within the spirit and scope of the claimed oral occlusion device and method of utilization.

I claim:

1. An oral occlusion device (10) for the treatment of disordered breathing, comprising;
a first surface (100) having a first surface length (102), a first surface width (103), a first surface thickness (104); and
a second surface (200), having a second surface length (202), a second surface width (203), a second surface thickness (204), and an adhesive area (210).

2. The device according to claim 1, wherein the second surface (200) has a central non-adhesive area (220) bounded by the adhesive area (210).

3. The device according to claim 2, wherein the central non-adhesive area (220) comprises a central non-adhesive dressing area (222).

4. The device according to claim 1, wherein the second surface (200) has at least one peripheral non-adhesive area (230).

5. The device according to claim 1, wherein the first surface further comprises at least one removal fixture (110).

6. The device according to claim 5, wherein the at least one removal fixture (110) comprises at least one humanly-grippable tab (112).

7. The device according to claim 1, wherein the device (10) is packaged prior to use with a protective cover (300) removably adhered to the second surface (200).

8. The device according to claim 3, wherein the central non-adhesive dressing area (222) further comprises a medicament.

9. An oral occlusion device (10) for the treatment of disordered breathing, comprising;
a first surface (100) having a first surface length (102), a first surface width (103), a first surface thickness (104); and
a second surface (200), having a second surface length (202), a second surface width (203), a second surface thickness (204), and an adhesive area (210), wherein the second surface to (200) has a central non-adhesive area (220) bounded by the adhesive area (210).

10. An oral occlusion device (10) for the treatment of disordered breathing, comprising;
a first surface (100) having a first surface length (102), a first surface width (103), a first surface thickness (104); and
a second surface (200), having a second surface length (202), a second surface width (203), a second surface thickness (204), and an adhesive area (210); and
the device is sized and shaped to be adhered to an area of lip skin within a vermillion border of a human user's lips.

11. The device according to claim 10, wherein the second surface (200) has a central non-adhesive area (220) bounded by the adhesive area (210).

12. The device according to claim 11, wherein the central non-adhesive area (220) comprises a central non-adhesive dressing area (222).

13. The device according to claim 10, wherein the second surface (200) has at least one peripheral non-adhesive area (230).

14. The device according to claim 10, wherein the first surface further comprises at least one removal fixture (110).

15. The device according to claim 14, wherein the at least one removal fixture (110) comprises at least one humanly-grippable tab (112).

16. The device according to claim 10, wherein the device (10) is packaged prior to use with a protective cover (300) removably adhered to the second surface (200).

17. The device according to claim 12, wherein the central non-adhesive dressing area (222) further comprises a medicament.
